(19) United States
(12) Patent Application Publication
(10) Pub. No.: US 2020/0085411 A1
(43) Pub. Date: Mar. 19, 2020

(54) METHOD, APPARATUS AND READABLE STORAGE MEDIUM FOR ACQUIRING AN IMAGE

(71) Applicant: CLOUDMINDS (SHENZHEN) HOLDINGS CO., LTD., Shenzhen (CN)

(72) Inventors: Lei Luo, Shenzhen (CN); Qingwei Ji, Shenzhen (CN)

(73) Assignee: CLOUDMINDS (SHENZHEN) HOLDINGS CO., LTD., Shenzhen (CN)

(21) Appl. No.: 16/572,837

(22) Filed: Sep. 17, 2019

(30) Foreign Application Priority Data
Sep. 17, 2018 (CN) 201811080993.2

(51) Int. Cl. A61B 8/08; A61B 8/00; G06T 7/00; G06T 7/70

(52) U.S. Cl. CPC A61B 8/5207; A61B 8/4245; G06T 2207/10136; G06T 7/70; G06T 7/0014

(57) ABSTRACT

The embodiments of the present disclosure relates to the field of telecommunication technology, and discloses a method, a device and a readable storage medium for acquiring an image. The method comprises: acquiring a first ultrasonic image of a first position of a detected object; and saving the first ultrasonic image at a second position in a three-dimensional model for the detected object that corresponds to the first position, wherein the three-dimensional model saves therein a historical ultrasonic image of the detected object acquired during one ultrasonic detection process. the method for acquiring an image according to the present embodiment expands the area of the ultrasonic image for a detected object acquired during one ultrasonic detection, by saving a ultrasonic image for a determined position of the detected object at a position in a three-dimensional model for the detected object corresponding to the determined position.

### CROSS-REFERENCE TO RELATED APPLICATION

This application claims the priority benefit of Chinese Patent Application No. 201811080993.2 filed on Sep. 17, 2018 and entitled “Method, apparatus and readable storage medium for aquiring an image”, the disclosure of which is incorporated by reference herein in its entirety.

### TECHNICAL FIELD

Embodiments of the present disclosure relate to the field of communication technology, and in particular, to a method, an apparatus and a readable storage medium for acquiring an image.

### BACKGROUND

As one of medical imaging technologies, ultrasonic imaging technology has been widely concerned and completely used in clinical diagnosis. In the ultrasonic imaging technology, a method of combining Augmented Reality (AR) technology with ultrasonic examination equipment is proposed, which includes obtaining images of parts to be examined through the ultrasonic examination equipment, transmitting the images to AR glasses and rendering the images in real time on a surface of a current correct position of a human body, so that a doctor may view impacts on organs at the examined parts in real time during a surgery and thus perform precise operations thereon.

The inventors have found that at least the following problems exist in the prior art: since the area of a probe of the ultrasonic examination equipment is small, only a small area corresponding to the probe could be viewed at a same time, and if the doctor desires to see a large range of blood vessels or arteries at a same time, he/she has to move the probe of the ultrasonic examination equipment slightly and continuously, thus the operational efficiency of the doctor is lowered.

### SUMMARY

One purpose of some embodiments of the present disclosure is to provide a method, a device and a readable storage medium for acquiring an image, so that the range of an ultrasonic image of a detected object saved in a three dimensional model for the detected object is expanded.

In order to solve the above technical problems, an embodiment of the present disclosure provides a method for acquiring an image, which is applied to a terminal. The method comprises: acquiring a first ultrasonic image of a first position of a detected object; and saving the first ultrasonic image at a second position in a three-dimensional model for the detected object that corresponds to the first position, wherein the three-dimensional model saves therein a historical ultrasonic image of the detected object acquired during one ultrasonic detection process.

An embodiment of the present disclosure further provides a terminal, comprising: at least one processor; and a memory communicatively coupled to the at least one processor; wherein the memory stores instructions executable by the at least one processor, the instructions being executed by the at least one processor to enable the at least one processor to implement the method for acquiring an image as described above.

An embodiment of the present disclosure further provides a computer readable storage medium storing a computer program, wherein the computer program is executed by a processor to implement the method for acquiring an image as described above.

Compared with the prior art, the method for acquiring an image according to the present embodiment expands the area of the ultrasonic image for a detected object acquired during one ultrasonic detection, by saving a ultrasonic image for a determined position of the detected object at a position in a three-dimensional model for the detected object corresponding to the determined position. During one ultrasonic detection, the determined position is determined by a position where the ultrasonic probe is located, and the ultrasonic images determined by the ultrasonic probe at respective positions are saved, thereby improving the operational efficiency of the user.

In addition, the method further comprises performing the following step before the acquiring a first ultrasonic image of a first position of a detected object: acquiring the three-dimensional model for the detected object.

In addition, the terminal is communicatively connected with an AR display device, and the AR display device is provided with an imaging device; and the acquiring the three-dimensional model for the detected object comprises: receiving an image of the detected object captured by the imaging device provided on the AR display device; and acquiring the three-dimensional model through three-dimensional modeling according to the image of the detected object.

In addition, the method further comprises performing the following step before the acquiring a first ultrasonic image of a first position of a detected object: acquiring a tracking result of tracking an ultrasonic probe by the imaging device provided on the AR display device, wherein the tracking result comprises a position of the ultrasonic probe; and if it is determined according to the tracking result that the position of the ultrasonic probe is changed, determining the changed position of the ultrasonic probe is as the first position of the detected object. In this implementation, the first position of the detected objected by obtaining the tracking result of tracking the ultrasonic probe through the imaging device and obtaining a change in position of the ultrasonic probe from the tracking result, so that the determination on the first position is more precise.

In addition, the acquiring a first ultrasonic image of a first position of a detected object specifically comprises: receiving a first reflected ultrasonic signal acquired by the ultrasonic probe at the first position of the detected object; and acquiring the first ultrasonic image according to the first reflected ultrasonic signal.

In addition, the method further comprises performing the following step after the saving the first ultrasonic image at a second position in a three-dimensional model for the detected object that corresponds to the first position: transmitting the three-dimensional model saved with the first ultrasonic image and the historical ultrasonic image to an AR display device, wherein the AR display device is configured to display the first ultrasonic image and the historical ultrasonic image saved in the three-dimensional model. Through displaying the three-dimensional model saved with the first ultrasonic image and the historical ultrasonic image on the human-computer interface, the user could perform corresponding operations on the human-computer interface according to the displayed image, thereby further improving the user's experience.

In addition, the method further comprises performing the following step before transmitting the three-dimensional model saved with the first ultrasonic image and the historical ultrasonic image to an AR display device: if it is determined that there is an overlapping region between the first ultrasonic image and the historical ultrasonic image, covering the overlapping region of the historical ultrasonic image with the overlapping region of the first ultrasonic image. In implementation, if there is an overlapping area between the first ultrasonic image and the historical ultrasonic image, the overlapping area of the historical ultrasonic image is covered with the overlapping area of the newly acquired ultrasonic image, so that the final ultrasonic image for each position is the acquired by the latest scanning of the ultrasonic probe, and thus the ultrasonic image finally obtained for a expanded range has a timeliness.

In addition, the method further comprises performing the following step after the saving the first ultrasonic image at a second position in a three-dimensional model for the detected object that corresponds to the first position: displaying the three-dimensional model saved with the first ultrasonic image and the historical ultrasonic image on a human-computer interface. In implementation, through displaying the three-dimensional model saved with the first ultrasonic image and the historical ultrasonic image on the human-computer interface, the user could perform corresponding operations on the human-computer interface according to the displayed image, thereby further improving the user's experience.

In addition, the method further comprises performing the following step after displaying the three-dimensional model saved with the first ultrasonic image and the historical ultrasonic image on a human-computer interface: if it is determined that an operational instruction is received from a user, performing marking in the three-dimensional model saved with the first ultrasonic image and the historical ultrasonic image according to the operational instruction. In implementation, through performing making in the three-dimensional model saved with the first ultrasonic image and the historical ultrasonic image, the user may analyze the three-dimensional model saved with the first ultrasonic image and the historical ultrasonic image according to the marking result.

In addition, the ultrasonic probe is provided with a positioning mark, and the tracking result is determined by tracking the positioning mark through the imaging device. In implementation, as a positioning mark is provided on the ultrasonic probe, it is easy for the imaging device to track and lock the ultrasonic probe, and thus the precision of the tracking result is improved.

In addition, the method further comprises performing the following step after the acquiring the three-dimensional model for the detected object: if it is determined, according to the image of the detected object captured by the imaging device, that a relative position between the AR display device and the detected object is changed, re-acquiring a three-dimensional model after the relative position is changed. In this implementation, after the relative position between the AR display device and the detected object is changed, through the re-acquired three-dimensional model, the position of the detected object in the ultrasonic image displayed by the AR display device is in consistent with the position of the detected object actually detected.

### BRIEF DESCRIPTION OF THE DRAWINGS

One or more embodiments are exemplarily described by using figures in the accompanying drawings corresponding thereto. The exemplary descriptions do not constitute a limitation on the embodiments. Elements with a same reference numeral in the accompanying drawings represent similar elements. Unless otherwise particularly stated, the figures in the accompanying drawings do not constitute a limitation.

FIG. 1 is a flow chart of a method for acquiring an image in an embodiment of the present application;

FIG. 2 is a flow chart of a method for acquiring an image in another embodiment of the present application;

FIG. 3 is a block diagram showing an apparatus for acquiring an image in yet another embodiment of the present application;

FIG. 4 is a block diagram showing an apparatus for acquiring an image in still another embodiment of the present application;

FIG. 5 is a schematic structural diagram of a terminal in another embodiment of the present application.

### DETAILED DESCRIPTION

To make the objective, technical solutions, and advantages of the present disclosure clearer, the embodiments of the present disclosure will be described in detail below with reference to the accompanying drawings. Those skilled in the art would appreciate that in various embodiments of the present application, numerous technical details are set forth to provide the reader with a better understanding of the present application. However, the technical solutions claimed in the present application may be implemented without these technical details and various changes and modifications made based on the following embodiments.

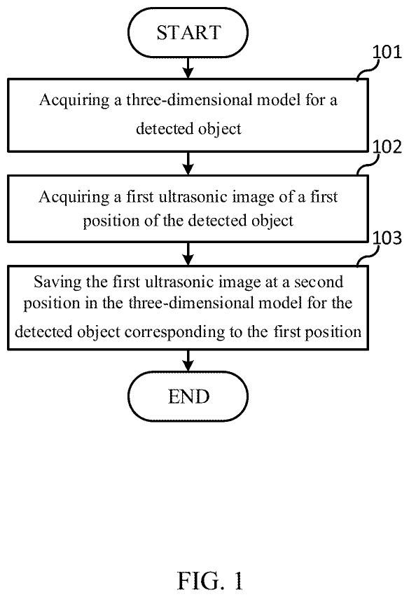

This embodiment of the present disclosure relates to a method for acquiring an image, which may be applied to a terminal device such as an ultrasonic detector. The specific process is shown in FIG. 1, which includes the following steps:

Step 101: acquiring a three-dimensional model of a detected object.

It should be noted that, in this embodiment, the terminal device is communicatively connected with an AR display device and a ultrasonic probe respectively. In practical applications, the AR display device is worn on eyes of a user and the position of the AR display device may be changed as the user's head moves. The AR display device may be provided with an imaging device, and the imaging device is generally disposed in front of the AR display device and captures an actual scene in front of the user's eyes as the user's head moves.

Specifically, when detecting the detected object, the imaging device provided on the AR display device captures an image of the detected object, and transmits the captured image of the detected object to a terminal, and the terminal receives the image captured by the imaging device provided on the AR display device. Since the received image is a two-dimensional planar image, after receiving the two-dimensional planar image of the detected object, the terminal may obtain a 3D model by performing a three-dimensional modeling according to the image of the detected object. For example, when the detected object is a abdomen of a certain patient, an image of the abdominal region captured by the imaging device provided on the AR display device is received, and a three-dimensional model for the abdominal region is obtained by three-dimensional modeling according to the acquired image of the abdominal region.

It is worth mentioning that when performing one ultrasonic detection, a historical ultrasonic image of the detected object acquired during the ultrasonic detection is stored in the three-dimensional model.

Step 102: acquiring a first ultrasonic image of a first position of the detected object.

It should be noted that before acquiring the first ultrasonic image of the first position of the detected object, it is necessary to determine the first position of the detected object. The AR display device may tracks the ultrasonic probe provided on the detected object while capturing an image of the detected object. The terminal obtains a tracking result of tracking the ultrasonic probe the imaging device provided on the AR display device, and the tracking result includes the position of the ultrasonic probe. If it is determined according to the tracking result that the position of the ultrasonic probe has changed, the changed position of the ultrasonic probe is determined as the first position of the detected object. That is to say, the first position of the detected object is not fixed, and if it is determined according to the tracking result that the current position of the ultrasonic probe is different from the position determined at previous time, the current position is determined as the first position of the detected object.

In practical applications, in order to make the imaging device more accurately track and lock the ultrasonic probe, a positioning mark may be provided on the ultrasonic probe, and the tracking result is determined by tracking the positioning mark through the imaging device.

Specifically, the specific way for acquiring the first ultrasonic image of the first position of the detected object includes: receiving a first reflected ultrasonic signal acquired by the ultrasonic probe at the first position of the detected object, and processing the acquired first reflected ultrasonic signal to obtain the first ultrasonic image; the first ultrasonic image obtained at this time has a transparent background. For example, if the first position of the detected object is a navel area, an image of an organ structure in the navel area of the abdomen is displayed in the first ultrasonic image.

Step 103: saving the first ultrasonic image at a second position in the three-dimensional model for the detected object that corresponds to the first position.

Specifically, the three-dimensional model for the detected object corresponds to the real detected object. For example, when a navel of the abdomen is determined as the first position, a position corresponding to the navel is found in the three-dimensional model, and the position is determined as the second position; and then the first ultrasonic image is saved at the second position in the three-dimensional model for the detected object.

It should be noted that, if it is determined that there is an overlapping area between the first ultrasonic image and the historical ultrasonic image, a method for saving images may be adopted such that the overlapping area of the newly obtained first ultrasonic image covers the corresponding area of the historical ultrasonic image. By covering the overlapping area of the historical ultrasonic image with the overlapping area of the newly acquired ultrasonic image, the final ultrasonic image for each position is the acquired by the latest scanning of the ultrasonic probe, so that the ultrasonic image finally obtained for a expanded range has a timeliness.

It is worth mentioning that after the first ultrasonic image is saved in the three-dimensional model, it is desirable that the user could view the ultrasonic image of the expanded range through the AR display device. Thus, the three-dimensional model saved with the first ultrasonic image and the historical ultrasonic image needs to be transmitted to the AR display device, and display the first ultrasonic image and the historical ultrasonic image by the AR display device according to corresponding positions in the three-dimensional model.

It should be noted that, if it is determined according to the image of the detected object captured by the imaging device that a relative position between the AR display device and the detected object is changed, a three-dimensional model after the change needs to be re-acquired. Through the re-acquired three-dimensional model, the position of the detected object in the ultrasonic image displayed by the AR display device is in consistent with the position of the detected object actually detected. For example, the AR display device and the detected object may have a vertical position relationship previously, if an angular offset is presented therebetween, a three-dimensional model after the position relationship is changed needs to be re-acquired, and the first ultrasonic image and the historical ultrasonic image are redisplayed on the AR display device according to the three-dimensional model acquired after the position relationship is changed.

Compared with the prior art, the method for acquiring an image according to the present embodiment expands the area of the ultrasonic image for a detected object acquired during one ultrasonic detection, by saving a ultrasonic image for a determined position of the detected object at a position in a three-dimensional model for the detected object corresponding to the determined position. During one ultrasonic detection, the determined position is determined by a position where the ultrasonic probe is located, and the ultrasonic images determined by the ultrasonic probe at respective positions are saved, thereby improving the operational efficiency of the user.

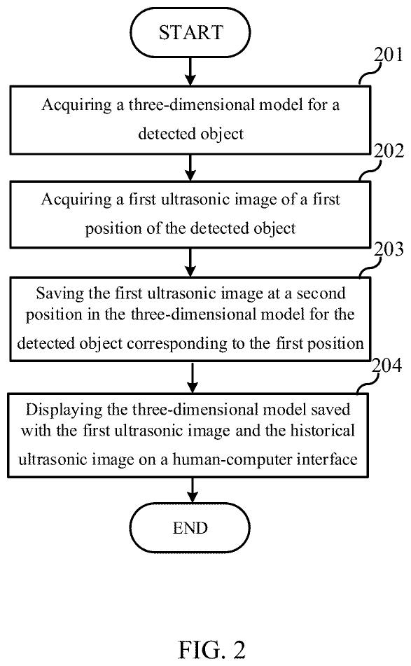

Another embodiment of the present disclosure relates to a method for acquiring an image. The embodiment is further improved on the basis of the embodiment described with reference to FIG. 1, and the specific improvement is that: after the first ultrasonic image is saved at the second position in the three-dimensional model for the detected object that corresponds to the first position, the 3D model is displayed on a human-computer interface. The flow of the method for acquiring an image in this embodiment is shown in FIG. 2. Specifically, in this embodiment, the method includes steps 201 to 204, and the steps 201 to 203 are substantially the same as the steps 101 to 103 in the embodiment described with reference to FIG. 1, and details thereof are not described herein again. The differences therebetween will be described as follows, and for the technical details that are not described in details in this embodiment, the method for acquiring an image provided by the embodiment described with reference to FIG. 1 may be referred to, and details thereof are not described herein again.

After step 201 to step 203, step 204 is performed.

At step 204, displaying the three-dimensional model saved with the first ultrasonic image and the historical ultrasonic image on a human-computer interface.

Specifically, by displaying the three-dimensional model saved with the first ultrasonic image and the historical ultrasonic image on the human-computer interface, the user may viewing the three-dimensional model and perform corresponding operations on the human-computer interface, for example, marking a lesion part of a certain organ of the abdomen, marking a part from where the tumor needs to be removed, and the like. The terminal, when determining that an operational instruction is received from the user, performs marking in the three-dimensional model saved with the first ultrasonic image and the historical ultrasonic image according to operational instruction.

Compared with the prior art, the method for acquiring an image according to the present embodiment expands the area of the ultrasonic image for a detected object acquired during one ultrasonic detection, by saving a ultrasonic image for a determined position of the detected object at a position in a three-dimensional model for the detected object corresponding to the determined position. During one ultrasonic detection, the determined position is determined by a position where the ultrasonic probe is located, and the ultrasonic images determined by the ultrasonic probe at respective positions are saved, thereby improving the operational efficiency of the user. Besides, through displaying the three-dimensional model saved with the first ultrasonic image and the historical ultrasonic image on the human-computer interface, the user could perform corresponding operations on the human-computer interface according to the displayed image, thereby further improving the user's experience.

Division of steps of the foregoing methods is made for the purpose of clear description, and during implementation, the steps may be combined into one step or some steps may be split into a plurality of steps. Provided that a same logical relationship is included, the division falls within the protection scope of this patent application. Unnecessary modifications or unnecessary designs added/introduced to an algorithm or a procedure also fall within the protection scope of this patent application as long as a core design of the algorithm or the procedure is not change.

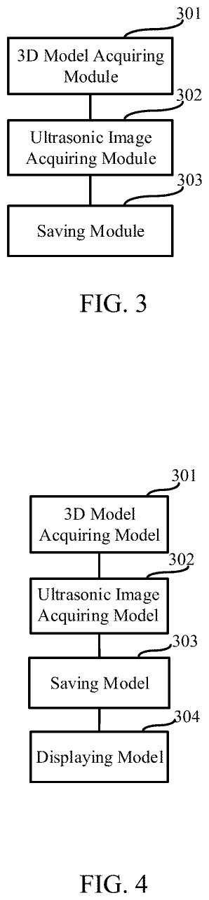

Yet another embodiment of the present disclosure relates to an apparatus for acquiring an image, and the specific structure is as shown in FIG. 3.

As shown in FIG. 3, the apparatus for acquiring an image includes a three-dimensional (3D) model acquiring module 301, an ultrasonic image acquiring module 302, and a saving module 303.

The three-dimensional model acquiring module 301 is configured to acquire a three-dimensional model of a detected object.

The ultrasonic image acquiring module 302 is configured to acquire a first ultrasonic image of a first position of the detected object.

The saving module 303 is configured to save the first ultrasonic image in a second position in the three-dimensional model for the detected object that corresponds to the first position.

It is not difficult to find that, this embodiment is an apparatus embodiment corresponding to the embodiment described with reference to FIG. 1, and thus it may be implemented in cooperation with the embodiment described with reference to FIG. 1. Related technical details mentioned in the embodiment described with reference to FIG. 1 still work in this embodiment, and details are not described herein again in order to avoid repetition. Correspondingly, the related technical details mentioned in this embodiment may also be applied to the embodiment described with reference to FIG. 1.

Still another embodiment of the present disclosure relates to an apparatus for acquiring an image. This embodiment is substantially the same as the embodiment described with reference to FIG. 3, and the specific structure is as shown in FIG. 4. The main improvement is that in the technical solution according to the fourth embodiment, a displaying module 304 is added to the apparatus for acquiring an image according to the embodiment described with reference to FIG. 3.

The three-dimensional model acquiring module 301 is configured to acquire a three-dimensional model of a detected object.

The ultrasonic image acquiring module 302 is configured to acquire a first ultrasonic image of a first position of the detected object.

The saving module 303 is configured to save the first ultrasonic image in a second position in the three-dimensional model for the detected object that corresponds to the first position.

The displaying module 304 is configured to display the three-dimensional model saved with the first ultrasonic image and the historical ultrasonic image on the human-computer interface.

It is not difficult to find that, this embodiment is an apparatus embodiment corresponding to the embodiment described with reference to FIG. 2, and thus it may be implemented in cooperation with the embodiment described with reference to FIG. 2. Related technical details mentioned in the embodiment described with reference to FIG. 2 still work in this embodiment, and details are not described herein again in order to avoid repetition. Correspondingly, the related technical details mentioned in this embodiment may also be applied to the embodiment described with reference to FIG. 2.

It should be noted that, the various modules in this embodiment are logical modules, and in an actual application, a logical unit may be a physical unit, or may be a part of a physical unit, or may be implemented by a combination of a plurality of physical units. In addition, to highlight a creative part of the present disclosure, units not closely related to the technical problem proposed in the present disclosure are not introduced in this embodiment. However, it does not indicate that there are no other units in this embodiment.

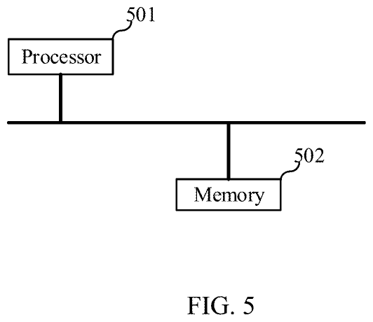

The present disclosure provides another embodiment, which relates to a terminal. As shown in FIG. 5, the terminal includes at least one processor 501; and a memory 502 communicatively connected with the at least one processor 501, where the memory 502 stores an instruction executable by the at least one processor 501, and the instruction is executed by the at least one processor 501, so that the at least one processor 501 is capable of implementing the method for acquiring an image according to the above embodiments.

In this embodiment, the processor 501 is exemplified by a Central Processing Unit (CPU), and the memory 502 is exemplified by a Random Access Memory (RAM). The processor 501 and the memory 502 may be connected by a bus or may be connected in other ways. In FIG. 5, the processor 501 and the memory 502 are connected by a bus, for example. The memory 502 is a non-volatile computer readable storage medium, and may be used for storing non-volatile software programs, non-volatile computer-executable programs and modules. Such as, a program for implementing a method for acquiring an image according to the embodiment of the present application is stored in the memory 502. The processor 501 performs various functional applications of the device and data processing by executing non-volatile software programs, instructions, and modules stored in the memory 502, that is, implementing the above-described method for acquiring an image.

The memory 502 may include a program storage area and a data storage area, wherein the program storage area may store an operating system and an application required by at least one function; the data storage area may store a list of options, and the like. Further, the memory may include a high speed random access memory, and it may also include a non-volatile memory such as at least one magnetic disk storage device, flash memory device, or other non-volatile solid state storage device. In some embodiments, the memory 502 may optionally include memories remotely located relative to the processor 501, the memories remotely located may be connected to external devices over a network. Such network may include the Internet, intranets, local area networks, mobile communication networks, and combinations thereof, but not limited thereto.

One or more program modules are stored in memory 502, which, when being executed by one or more processors 501, perform the method for acquiring an image according to any of the above-described method embodiments.

The above-mentioned products may implement the method provided by the embodiments of the present application, and thus have corresponding functional modules for implementing the method and the beneficial effects thereof. For technical details not described in the this embodiments, the description on the methods according to the embodiments of the present application may be referred to.

The present disclosure provides another embodiment, which relates to a computer readable storage medium having stored therein a computer program. When the computer program is executed by a processor, the foregoing method for acquiring an image according to any of the embodiments of the present application is implemented.

A person skilled in the art may understand that all or some steps in the foregoing method embodiments may be completed by related hardware instructed through a program. The program is stored in one storage medium, and includes several instructions to cause a device (which may be a single-chip microcomputer, a chip, or the like) or the processor to perform all or some steps of the methods in the embodiments in the present disclosure. The foregoing storage medium includes various media that can store program code, for example: a USB flash drive, a removable hard disk, a read-only memory (ROM,), a random access memory (RAM), a magnetic disk, or an optical disc.

A person of ordinary skill in the art may understand that the foregoing embodiments are specific embodiments for implementing the present disclosure, and various modifications may be made to the embodiments in forms and in details during actual application without departing from the spirit and scope of the present disclosure.

1. A method for acquiring an image, wherein, the method is applied to a terminal, the method comprising:
acquiring a first ultrasonic image of a first position of a detected object; and
saving the first ultrasonic image at a second position in a three-dimensional model for the detected object that corresponds to the first position, wherein the three-dimensional model saves therein a historical ultrasonic image of the detected object acquired during one ultrasonic detection process.

2. The method for acquiring an image according to claim 1, further comprising performing the following step before the acquiring a first ultrasonic image of a first position of a detected object:
acquiring the three-dimensional model for the detected object.

3. The method for acquiring an image according to claim 2, wherein the terminal is communicatively connected with an AR display device, and the AR display device is provided with an imaging device;
the acquiring the three-dimensional model for the detected object comprises:
receiving an image of the detected object captured by the imaging device provided on the AR display device; and
acquiring the three-dimensional model through three-dimensional modeling according to the image of the detected object.

4. The method for acquiring an image according to claim 3, further comprising performing the following step before the acquiring a first ultrasonic image of a first position of a detected object:
acquiring a tracking result of tracking an ultrasonic probe by the imaging device provided on the AR display device, wherein the tracking result comprises a position of the ultrasonic probe; and
if it is determined according to the tracking result that the position of the ultrasonic probe is changed, determining the changed position of the ultrasonic probe as the first position of the detected object.

5. The method for acquiring an image according to claim 4, wherein, the acquiring a first ultrasonic image of a first position of a detected object comprises:
receiving a first reflected ultrasonic signal acquired by the ultrasonic probe at the first position of the detected object; and
acquiring the first ultrasonic image according to the first reflected ultrasonic signal.

6. The method for acquiring an image according to claim 1, further comprising performing the following step after the saving the first ultrasonic image at a second position in a three-dimensional model for the detected object that corresponds to the first position:
transmitting the three-dimensional model saved with the first ultrasonic image and the historical ultrasonic image to an AR display device, wherein the AR display device is configured to display the first ultrasonic image and the historical ultrasonic image saved in the three-dimensional model.

7. The method for acquiring an image according to claim 6, further comprising performing the following step before transmitting the three-dimensional model saved with the first ultrasonic image and the historical ultrasonic image to an AR display device:
if it is determined that there is an overlapping region between the first ultrasonic image and the historical ultrasonic image, covering the overlapping region of the historical ultrasonic image with the overlapping region of the first ultrasonic image.

8. The method for acquiring an image according to claim 1, further comprising performing the following step after the saving the first ultrasonic image at a second position in a three-dimensional model for the detected object that corresponds to the first position:
displaying the three-dimensional model saved with the first ultrasonic image and the historical ultrasonic image on a human-computer interface.

9. The method for acquiring an image according to claim 8, further comprising performing the following step after displaying the three-dimensional model saved with the first ultrasonic image and the historical ultrasonic image on a human-computer interface:
if it is determined that an operational instruction is received from a user, performing marking in the three-dimensional model saved with the first ultrasonic image and the historical ultrasonic image according to the operational instruction.

10. The method for acquiring an image according to claim 4, wherein the ultrasonic probe is provided with a positioning mark, and the tracking result is determined by tracking the positioning mark through the imaging device.

11. The method for acquiring an image according to claim 3, further comprising performing the following step after the acquiring the three-dimensional model for the detected object:
if it is determined, according to the image of the detected object captured by the imaging device, that a relative position between the AR display device and the detected object is changed, re-acquiring a three-dimensional model after the relative position is changed.

12. A terminal, comprising:
at least one processor; and
a memory communicatively coupled to the at least one processor; wherein
the memory stores instructions executable by the at least one processor, the instructions being executed by the at least one processor to enable the at least one processor to implement the following steps:
acquiring a first ultrasonic image of a first position of a detected object; and
saving the first ultrasonic image at a second position in a three-dimensional model for the detected object that corresponds to the first position, wherein the three-dimensional model saves therein a historical ultrasonic image of the detected object acquired during one ultrasonic detection process.

13. The terminal according to claim 12, wherein, the instruction further enables the at least one processor to implement the following step before the acquiring a first ultrasonic image of a first position of a detected object:
acquiring the three-dimensional model for the detected object.

14. The terminal according to claim 13, wherein, the terminal is communicatively connected with an AR display device, and the AR display device is provided with an imaging device;
the acquiring the three-dimensional model for the detected object comprises:
receiving an image of the detected object captured by the imaging device provided on the AR display device; and
acquiring the three-dimensional model through three-dimensional modeling according to the image of the detected object.

15. The terminal according to claim 14, wherein, the instruction further enables the at least one processor to implement the following step before the acquiring a first ultrasonic image of a first position of a detected object:
acquiring a tracking result of tracking an ultrasonic probe by the imaging device provided on the AR display device, wherein the tracking result comprises a position of the ultrasonic probe; and
if it is determined according to the tracking result that the position of the ultrasonic probe is changed, determining the changed position of the ultrasonic probe as the first position of the detected object.

16. The terminal according to claim 15, wherein, the acquiring a first ultrasonic image of a first position of a detected object comprises:
receiving a first reflected ultrasonic signal acquired by the ultrasonic probe at the first position of the detected object; and
acquiring the first ultrasonic image according to the first reflected ultrasonic signal.

17. The terminal according to claim 12, wherein, the instruction further enables the at least one processor to implement the following step after the saving the first ultrasonic image at a second position in a three-dimensional model for the detected object that corresponds to the first position:
transmitting the three-dimensional model saved with the first ultrasonic image and the historical ultrasonic image to an AR display device, wherein the AR display device is configured to display the first ultrasonic image and the historical ultrasonic image saved in the three-dimensional model.

18. The terminal according to claim 17, wherein, the instruction further enables the at least one processor to implement the following step before transmitting the three-dimensional model saved with the first ultrasonic image and the historical ultrasonic image to an AR display device:
if it is determined that there is an overlapping region between the first ultrasonic image and the historical ultrasonic image, covering the overlapping region of the historical ultrasonic image with the overlapping region of the first ultrasonic image.

19. The terminal according to claim 14, further comprising performing the following step after the acquiring the three-dimensional model for the detected object:
if it is determined, according to the image of the detected object captured by the imaging device, that a relative position between the AR display device and the detected object is changed, re-acquiring a three-dimensional model after the relative position is changed.

20. A computer readable storage medium storing a computer program, wherein the computer program is executed by a processor to implement the following steps:
acquiring a first ultrasonic image of a first position of a detected object; and
saving the first ultrasonic image at a second position in a three-dimensional model for the detected object that corresponds to the first position, wherein the three-dimensional model saves therein a historical ultrasonic image of the detected object acquired during one ultrasonic detection process.
